(19) United States
(12) Patent Application Publication
(10) Pub. No.: US 2024/0368536 A1
(43) Pub. Date: Nov. 7, 2024

(54) INCREASING GROWTH OF A CO2 FIXING THERMOPHILE BACTERIUM

(71) Applicant: DANMARKS TEKNISKE UNIVERSITET, Kongens Lyngby (DK)

(72) Inventors: Alex Toftgaard Nielsen, Kongens Lyngby (DK); Torbjørn Ølshøj Jensen, Kongens Lyngby (DK); Amalie Melton Axelsen, Kongens Lyngby (DK); Stephanie Redl, Kongens Lyngby (DK); Sebastian Sven Brøndum, Kongens Lyngby (DK)

(21) Appl. No.: 18/688,433

(22) PCT Filed: Sep. 7, 2022
(86) PCT No.: PCT/EP2022/074870

§ 371 Date: Mar. 1, 2024

(30) Foreign Application Priority Data
Sep. 7, 2021 (EP) 21195239.5

(51) Int. Cl. C12N 1/20; C12N 15/74; C12P 7/00; C12R 1/145

(52) U.S. Cl. CPC C12N 1/205; C12N 15/74; C12P 7/00; C12N 2510/02; C12N 2511/00; C12R 2001/145

(57) ABSTRACT

Provided are methods for increasing the growth of Moorella species bacteria, genetically modified bacteria derived from such methods, and use of such bacteria for metabolizing a carbon-containing substrate, optionally in the production of a biochemical.

### FIELD OF THE INVENTION

The present invention relates to the use of thermophilic bacteria for fixation of CO₂ and production of biochemicals and to methods of increasing the growth of such bacteria by genetic modification, leading to an increased efficiency of CO₂ fixation.

### BACKGROUND OF THE INVENTION

Bulk chemicals, on the scale of millions of tons, are produced unsustainably, by cracking of fossil fuels. Simultaneously, humankind is emitting more than 40 Gigaton of CO₂ into the atmosphere every year, leading to a changing climate and the threatening effects of rising temperatures. Technologies that offer to capture industrial CO₂ emissions and convert the carbon into value are emerging. However, efficiency and feasibility are limiting the implementation. Traditional technologies for CO₂ capture include filters, planting trees, or growing algae. Filters require expensive catalysts, which are sensitive to impurities in the CO₂ gas, while planting trees and growing algae has extremely low land-area efficiency.

To develop processes meeting these limitations, application of bacteria and in particular bacteria operating at high temperatures is foreseen of great importance. The high cultivation temperature reduces risk of contamination with unwanted microorganisms. Fermentations typically require large amounts of cooling water. For fermentations using thermophilic bacteria, this requirement does not apply. Overall, thermophilic fermentation processes have characteristics which lead to significantly lower capital and operational expenditures when compared to other bio-based production processes.

Acetogenic bacteria are a group of bacteria growing with CO₂ (or CO) as sole carbon source. The growth of acetogenic bacteria is directly linked to the fixation of CO₂. One organism, Moorella thermoacetica, has properties that makes it interesting for fixing CO₂ from industrial points of view. Although CO₂ fixation is very efficient in this organism, the growth-rate is limiting. Strains with higher growth-rates will be highly beneficial in making CO₂ fixation more efficient. In the industrial production there will be fluctuations in the gas supply as well as gradients in the bioreactor. M. thermoacetica is known to either die or sporulate if nutrients or substrate are limited. This will result in inactive subcultures, decreasing the overall efficiency significantly. Developing cells capable of being viable for a longer period or under more stressful conditions and recover faster (when nutrient or substrate becomes available) will benefit the efficiency of the process. WO 2011/019717 A1 (Mascoma Corp.) relates to vectors encoding selectable markers and their use in, e.g., replacing target genes such as, e.g., spo0A, with such markers in thermophilic bacterial host cells. WO 2020/157487 A2 (Univ. Nottingham) relates to a genetic construct for use in controlling gene expression of e.g., Spo0A, in a spore-forming cell.

Stage 0 sporulation protein A homolog (Spo0A) is a protein involved in regulating bacterial sporulation. Spo0A binds to DNA and controls the expression of many genes (Molle et al., Mol. Microbiol.; 50:1683-1701 2003). It activates the sporulation cascade in different genera including Bacilli and Clostridia. Deletion of the spo0A gene in Bacillus subtilis has been reported to prevent sporulation (Spigelman et al., J. Bacteriol.; 172:5011-5019 1990).

HTH-type transcriptional regulator SinR (SinR) has been reported to function both as a negative and positive regulator of developmental processes that are induced at the end of vegetative growth in response to nutrient depletion. For example, it acts as a repressor of Spo0A. SinR tetramers act as transcriptional repressors of matrix genes during vegetative growth, whereas, during stationary phase, SinR monomers form a complex with either SinI or SlrR. SinI is an anti-repressor and can sequester SinR, while SlrR-SinR complexes release repression of the matrix operons and instead repress genes needed for planktonic growth (Kearns et al., Mol. Microbiol.; 55:739-749 2005, Chai et al., Mol. Microbiol.; 74:876-887 2009, Chai et al., Genes Dev.; 24:754-765 2010).

### SUMMARY OF THE INVENTION

It has been found by the present inventors that the growth of a Moorella species bacteria can be increased by genetic modifications of the genes encoding SinR and Spo0A. Accordingly, the invention generally relates to methods of enhancing the growth of Moorella species bacteria, thereby increasing their efficiency of CO₂ fixation and biochemical production.

So, in a first aspect the present invention relates to a method for increasing the growth-rate of a bacterium belonging to a Moorella species, comprising introducing one or more genetic modifications into the bacterium to reduce or abolish the expression and/or activity of Stage 0 sporulation protein A homolog (Spo0A) in the bacterium.

In some embodiments, the one or more genetic modifications comprise a genetic modification which reduces or abolishes the expression of Spo0A protein in the bacterium.

In some embodiments, the spo0A gene is deleted.

In some embodiments, the method further comprises introducing one or more genetic modifications into the bacterium to express a variant of SinR in the bacterium, wherein the SinR variant has at least 90% sequence identity with SEQ ID NO: 2 and comprises an amino acid other than V at the position corresponding to position 198 in SEQ ID NO: 2, preferably wherein said amino acid is F, I, Y, or W, more preferably wherein said amino acid is F, wherein the SinR variant provides for a decreased duration of the lag phase and/or an increased growth-rate of the bacterium as compared to SEQ ID NO: 2.

In a second aspect the present invention relates to a method for decreasing the duration of a lag phase and/or for increasing the growth-rate of a bacterium belonging to a Moorella species, comprising introducing one or more genetic modifications into the bacterium to express a variant of HTH-type transcriptional regulator SinR (SinR) in the bacterium, wherein the SinR variant has at least 90% sequence identity with SEQ ID NO: 2 and comprises an amino acid other than valine (V) at the position corresponding to position 198 in SEQ ID NO: 2, wherein the SinR variant provides for a decreased duration of the lag phase and/or an increased growth-rate of the bacterium as compared to SEQ ID NO: 2.

In some embodiments, the amino acid at the position corresponding to position 198 in SEQ ID NO: 2 is phenylalanine (F), isoleucine (I), tyrosine (Y), or tryptophan (W).

In some embodiments, the amino acid at the position corresponding to position 198 in SEQ ID NO: 2 is F. In some embodiments of the first and second aspects, the Moorella species is selected from (a) Moorella thermoacetica; (b) Moorella thermoautotrophica; (c) a bacterial strain having an average nucleotide identity based on MUMmer alignment (ANIm) score of at least about 96.5% compared to M. thermoacetica strain DSM 512^{T}; (d) a bacterial strain having an average nucleotide identity based on MUMmer alignment (ANIm) score of at least about 96.5% compared to M. thermoacetica strain DSM 2955^{T}; and a combination of (a) and (b); (a) and (c); (a) and (d); (a), (b) and (c), or all of (a) to (d).

In a third aspect, the present invention relates to a genetically modified bacterium obtained or obtainable by the method according to embodiments of the first or second aspect.

In a fourth aspect, the present invention relates to a bacterium belonging to the M. thermoacetica and/or M. thermoautotrophica species, wherein the bacterium has been genetically modified to reduce or abolish the expression and/or activity of Spo0A in the bacterium, wherein the reduced expression and/or activity is relative to its expression and/or activity in wildtype M. thermoacetica and/or M. thermoautotrophica.

In a fifth aspect, the present invention relates to a bacterium belonging to the M. thermoacetica and/or M. thermoautotrophica species, wherein the bacterium has been genetically modified to comprise a transgene encoding a variant of SinR, wherein the SinR variant has at least 90% sequence identity with SEQ ID NO: 2 and comprises an amino acid other than V at the position corresponding to position 198 in SEQ ID NO: 2, and wherein the SinR variant provides for a decreased duration of a lag phase and/or an increased growth-rate of the bacterium as compared to SEQ ID NO: 2. Optionally, the bacterium is of the M. thermoacetica ATCC 39073 strain or a strain derived therefrom, such as the M. thermoacetica 39073-HH strain.

In a sixth aspect, the present invention relates to a bacterium belonging to the M. thermoacetica and/or M. thermoautotrophica species, wherein the bacterium
(a) comprises a variant of SinR having at least 90% sequence identity with SEQ ID NO: 2 and comprising an amino acid other than V at the position corresponding to position 198 in SEQ ID NO: 2, wherein the SinR variant provides for a decreased duration of a lag phase and/or an increased growth-rate of the bacterium as compared to SEQ ID NO: 2, and
(b) has a reduced or abolished expression and/or activity of Spo0A, wherein the reduced expression and/or activity is relative to its expression and/or activity in wildtype M. thermoacetica and/or M. thermoautotrophica.

In some embodiments of the fourth and sixth aspects, the spo0A gene is deleted.

In some embodiments of the fifth and sixth aspects, the amino acid at the position corresponding to position 198 in SEQ ID NO: 2 is F.

In a seventh aspect, the present invention relates to use of a bacterium according to any one of aspects 3-6 for metabolizing a carbon-containing substrate, optionally in the production of a biochemical.

In some embodiments;
i) the carbon-containing substrate is CO and/or CO₂,
ii) the biochemical is selected from C1-C4 alcohols, C1-C4 ketones, C1-C4 aldehydes, C1-C4 carboxylic acids, and any mixtures thereof, or
iii) both i) and ii).

In some embodiments of the first to seventh aspects, the bacterium is of the M. thermoacetica ATCC 39073 strain or a strain derived therefrom, such as the M. thermoacetica 39073-HH strain.

### FIGURE LEGENDS

FIG. 1: Schematic illustration of a growth curve of a bacterial culture as determined by optical density (OD) measurements. The growth of a bacterial culture can be divided into four phases: lag phase, log phase, stationary phase, and death phase.

FIG. 2: Plasmid map of the spo0A-knock-out plasmids.

FIG. 3: Growth curves of WT and Δspo0A strains as a function of time in hours (h).
A; shows the individual measurements of the triplicate cultivations.
B; average growth curves, the light-colored patterns show the standard deviation.
C; same as B but optical density in logarithmic scale.

FIG. 4: Structural analysis of SinR-SinI complex from Bacillus.
A; SinR-SinI complex from Bacillus (PDB ID: 1b0n). The HTH domain from SinR is shown without any patterns, the oligomerization domain is shown by a pattern with circles, and SinI by a pattern with pentamer.
B; pattern scheme as in A, with the sidechains of T60 and L61 shown in stick representation having pattern of small solid triangles.
C; Zoom in on L61, and visualization of the L to F mutation. The proposed structure of the phenylalanine is shown in grey stick representation. The visible disks and patterns indicate pairwise overlap of atomic van der Waals radii. Short lines or small disks are shown when atoms are almost in contact or slightly overlapping. Large disks with crosses indicate significant van der Waals overlap. Everything else lies between those extremes.
D; Zoom in on T60, and visualization of the T to F mutation. Left: hydrogen bonds between T60 in SinR and E14 in SinI. Middle: T to F mutation, with the phenylalanine in the most common rotamer. Right: T to F mutation, with the phenylalanine in the most favourable rotamer. Disk and patterns are as indicated in C.

### DETAILED DISCLOSURE OF THE INVENTION

### Definitions

As used herein, the term “Moorella species” refers to any member of the group of species classified as belonging to the bacterial genus Moorella, belonging to the phylum Firmicutes. Moorella species are typically thermophilic, anaerobic and endospore-forming and may, for example, be isolated from hot springs. A non-limiting list of Moorella species can be found at the National Center for Biotechnology Information (World-Wide Web (www) address ncbi.nlm.nih.gov/Taxonomy/Browser/wwwtax.cgi?id=44260; accessed on 1 Jul. 2021, hereby incorporated by reference in its entirety) and elsewhere herein.

Particularly preferred are the acetogenic (gas-fermenting) species Moorella thermoacetica (M. thermoacetica), a species previously known as Clostridium thermoaceticum, and Moorella thermoautotrophica (M. thermoautotrophica) and any and all strains deriving therefrom, including strains isolated in a laboratory environment or isolated from natural sources. Although M. thermoacetica and M. thermoautotrophica are often considered as two different species, genomic comparisons have shown that M. thermoautotrophica strains may be re-classified as strains of M. thermoacetica (Redl et al., Front. Microbiol.; 10:3070 2020). Therefore, as used herein, M. thermoacetica may both refer to strains of bacteria commonly classified as M. thermoacetica and strains of bacteria that by a genetic analysis can be classified as M. thermoacetica strains, such as strains of M. thermoautotrophica. A method for determining whether a bacterial strain belongs to the M. thermoacetica species is described below. Non-limiting examples of M. thermoacetica strains include M. thermoacetica ATCC 39073, M. thermoacetica ATCC 39073-HH (Genbank accession number CP031054, preferably version CP031054.1), and M. thermoacetica Y72. As used herein, “wildtype M. thermoacetica and/or M. thermoautotrophica” refers to any naturally occurring strain of M. thermoacetica and/or M. thermoautotrophica. For example, typically the genome of a wildtype M. thermoacetica comprises a spo0A gene, a gene encoding a SinR protein (preferably with a valine in the amino acid position corresponding to 198 in SEQ ID NO: 2), or both.

As used herein, the term “SinR” or “HTH-type transcriptional regulator SinR” includes all variants of SinR without limitation to variants encoded by Moorella species bacteria. An example of a variant of SinR encoded by Moorella thermoacetica is the protein with UniProt ID: AOA5B7YPR1 (SEQ ID NO: 2), see Table 1. As used herein, the term “SinR” refers to a protein which has at least 80%, such as 85%, such as 90%, such as 91%, such as 92%, such as 93%, such as 94%, such as 95%, such as 96%, such as 97%, such as 98%, and such as 99%, sequence identity to SEQ ID NO: 2. Preferably, prior to any genetic modifications according to the methods described herein, the Moorella species cell to be modified comprises a native SinR protein, which protein preferably comprises a valine at the amino acid position corresponding to position 198 in SEQ ID NO: 2. Preferably, SinR of M. thermoacetica is encoded by the gene with European Nucleotide Archive (ENA) locus tag MothHH_01753 (SEQ ID NO: 1), see Table 1.

As used herein, the term “Spo0A” or “Stage 0 sporulation protein A homolog” refers to the endogenous protein of the relevant Moorella species. An example of Spo0A is M. thermoacetica Spo0A with UniProt ID: AOA5B7YPG0 (SEQ ID NO: 4), please refer to Table 1. Another example of Spo0A is M. thermoacetica Spo0A with UniProt ID: AOA1D7XBE2. As used herein, the term “Spo0A” refers to a protein which has at least 80%, such as 85%, such as 90%, such as 91%, such as 92%, such as 93%, such as 94%, such as 95%, such as 96%, such as 97%, such as 98%, and such as 99%, sequence identity to SEQ ID NO: 4. Preferably, Spo0A of M. thermoacetica is encoded by the gene with ENA locus tag MothHH_01617 (SEQ ID NO: 3), see Table 1.

The term “gene” refers to a nucleic acid sequence that encodes a cellular function, such as a protein, optionally including regulatory sequences preceding (5′ non-coding sequences) and following (3′ non-coding sequences) the coding sequence. A “transgene” is a gene, native or heterologous, that has been introduced into a cell, by a genetic engineering technique, such as by transformation. Gene names are herein set forth in italicised text with a lower-case first letter (e.g., spo0A) whereas protein names are set forth in normal text with a capital first letter (e.g., Spo0A).

TABLE 1
Spo0A and SinR in Moorella thermoacetica

| Gene name-ENA | | |
|---|---|---|
| locus tag, genomic | Protein name- | |
| location | UniProtKB reference | Sequences* |
| sinR_1- | HTH-type transcriptional | Gene: 903 bp |
| MothHH_01753, | regulator SinR- | (SEQ ID NO: 1) |
| CP031054 region: | A0A5B7YPR1 | Protein: 300 aa |
| 1679119..1680021 | (A0A5B7YPR1_MOOTH) | (SEQ ID NO: 2) |
| spo0A- | Stage 0 sporulation | Gene: 756 bp |
| MothHH_01617, | protein A homolog- | (SEQ ID NO: 3) |
| CP031054 region: | A0A5B7YPG0 | Protein: 251 aa |
| 1565989..1566744 | (A0A5B7YPG0_MOOTH) | (SEQ ID NO: 4) |

*bp = base pairs, aa = amino acids

As used herein, a “genetic modification” refers to the introduction of a genetically inherited change in the host cell genome. Examples of changes include mutations in genes and regulatory sequences, mutations in coding and non-coding DNA sequences. “Mutations” include deletions, substitutions and insertion of nucleic acids or nucleic acid fragments in the genome.

A “variant” of a parent or reference protein comprises one or more mutations, such as amino acid substitutions, insertions and deletions, as compared to the parent or reference protein. Typically, the variant has a high sequence identity to the amino acid sequence of the parent or reference protein, e.g., at least about 70%, such as at least about 80%, such as at least 84%, such as at least 85%, such as at least 87%, such as at least about 90%, such as at least about 93%, such as at least about 95%, such as at least about 96%, such as at least about 97%, such as at least about 98%, such as at least about 99%, over at least the functionally or catalytically active portion, optionally over the full length.

Unless otherwise stated, “sequence identity”, as used for amino acid sequences herein, is determined by comparing two optimally aligned sequences of equal length according to the following formula: (N_{ref}-N_{dif})·100/N_{ref}, wherein N_{ref} is the number of residues in one of the two sequences and N_{d}if is the number of residues which are non-identical in the two sequences when they are aligned over their entire lengths and in the same direction. Hence, the amino acid sequence GSTDYTQNWA (SEQ ID NO: 19) will have a sequence identity of 80% with the sequence GSTGYTQAWA (SEQ ID NO: 20; n_{dif}=2 and n_{ref}=10).

The sequence identity can be determined by conventional methods, e.g., Smith and Waterman (Adv. Appl. Math.; 2:482 1981), by the ‘search for similarity’ method of Pearson and Lipman (Proc. Natl. Acad. Sci. USA; 85:2444 1988), using the CLUSTAL W algorithm of Thompson et al. (Nucleic Acids Res.; 22:467380 1994), by computerized implementations of these algorithms (GAP, BESTFIT, FASTA, and TFASTA in the Wisconsin Genetics Software Package, Genetics Computer Group), or the Needleman-Wunsch algorithm (Needleman and Wunsch, J. Mol. Biol.; 48:443-453 1970) as implemented in the Needle program of the EMBOSS package (EMBOSS: The European Molecular Biology Open Software Suite, Rice et al., Trends Genet.; 16:276-277 2000), e.g., as provided at the European Bioinformatics Institute website (www.ebi.ac.uk). The BLAST algorithm (Altschul et al., Mol. Biol.; 215:403-410 1990), for which software may be obtained through the National Center for Biotechnology Information (www.ncbi.nlm.nih.gov/), may also be used. When using any of the mentioned algorithms, the default parameters for “Window” length, gap penalty, etc., may be used.

A residue in one amino acid sequence which “corresponds to” a specific reference residue in a reference amino acid sequence is the residue which aligns with the reference residue, e.g., as determined by use of sequence alignment software described in the preceding paragraph.

The term “expression”, as used herein, refers to the process in which a gene is transcribed into mRNA, and may optionally include the subsequent translation of the mRNA into an amino acid sequence, i.e., a protein or polypeptide.

As used herein, “reduced expression” of a gene in a host cell means that the levels of the mRNA or protein encoded by the gene are significantly reduced in the host cell, typically by at least 25%, such as at least 50%, such as at least 75%, such as at least 90%, such as at least 95%, as compared to a control. Typically, when the reduced expression is obtained by a genetic modification in the host cell, the control is the unmodified host cell.

By “abolished expression” of a gene in a host cell is meant that mRNA or protein encoded by that gene is essentially absent, absent or undetectable in the host cell.

The term “knock-down”, as used herein, refers to any of a range of techniques resulting in reduced expression of a gene in a host cell, such as introduction of a mutation in a promoter.

The term “knock-out”, as used herein, refers to any of a range of techniques resulting in abolished expression of a gene in a host cell, such as introduction of a mutation in, or deletion of, the gene. The term “deletion”, as used herein, refers to a partial or complete removal of the coding sequence of a gene, which either results in abolished expression of that gene or in the expression of a non-functional gene product.

The term “activity” or “function”, as used herein and when referring to the activity or function of a protein, can, when nothing more is specified, mean any activity or function of that protein—such as catalytic activity, binding activity, repressor activity, etc.

As used herein, “reduced activity” of a protein in a host cell means that one or more specific activities of that protein are significantly reduced in the host cell, typically by at least 25%, such as at least 50%, such as at least 75%, such as at least 90%, such as at least 95%, as compared to a control. Typically, when the reduced activity is obtained by a genetic modification in the host cell, the control is the unmodified host cell. By “abolished activity” of a protein in a host cell is meant that a one or more specific activities of that protein are essentially absent, absent or undetectable in the host cell.

Genetic modifications resulting in reduced or abolished activity of a target protein can include a mutation or deletion in the coding sequence of that protein which results in the expression of non-functional or less functional protein. Furthermore, genetic modifications resulting in reduced or abolished expression and/or activity of a target gene, as used herein, may be indirect, meaning that they are not genetic modifications in the gene itself. Such genetic modifications may for example include the introduction of a nucleic acid sequence that reduces the expression of the target gene, e.g., a repressor that inhibits expression of the target gene.

Standard recombinant DNA and molecular cloning techniques useful for carrying out embodiments of the present invention are well known in the art and are described by, e.g., Sambrook, J., Fritsch, E. F., and Maniatis, T. (2012). Molecular cloning: A laboratory manual, 4th ed. Cold Spring Harbor Laboratory: Cold Spring Harbor, New York, and by Silhavy, T. J., Bennan, M. L., and Enquist, L. W. (1984). Experiments with gene fusions. Cold Spring Harbor Laboratory: Cold Spring Harbor, New York. Techniques for targeted genome editing, such as knock-out of a target gene in a bacterial genome, include Clustered regularly interspaced short palindromic repeats (CRISPR)-based systems, such as CRISPR-Cas9.

The “growth-rate” of a bacterium, as used herein, is a measure reflecting the number of cell divisions per time unit. It can be calculated based on optical density (OD) measurements of the bacterial culture at 600 nm, where the growth-rate can be expressed as the change in OD per time unit, e.g. per hour.

As used herein, the term “lag phase”, when referring to the lag phase of a bacterium, means the first of four phases of bacterial growth: lag phase, log phase, stationary phase, and death phase. The lag phase is the phase where bacteria typically adapt themselves to new external conditions before they start replicating (enter log phase). Non-limiting examples of new external conditions include inoculation into new medium and addition of nutrients to an existing culture, e.g., a carbon source. During the lag phase, cell division is usually low or non-existent.

As used herein, “metabolizing” means the consumption of a substrate in one or more metabolic processes, optionally catalysed by one or more enzymes.

The term “substrate”, as used herein, refers to a molecule upon which an enzyme acts to form a product, converting the substrate in the process. When used in relation to a biosynthetic pathway, the term “substrate” refers to the molecule(s) upon which the first enzyme of the referenced pathway acts. A “carbon-containing substrate” is a substrate containing at least one carbon atom, such as CO or CO₂.

As used herein, a “biochemical” means a molecule which can be produced by a biological process. In the context of the present invention, Moorella species bacteria can be used to produce biochemicals, either by the actions of their natural, endogenous enzymes, or after a genetic modification; such as insertion of one or more transgenes encoding specific enzymes suitable for producing a biochemical of interest.

### Specific Embodiments of the Invention

As described in Example 1, the growth-rate of M. thermoacetica was increased by deletion of the gene encoding Spo0A (Example 1; FIG. 3 and Table 3).

Regulation of gene expression for controlling cellular growth-rate is both complex and delicately tuned, usually with many genes/proteins involved. Here, however, the deletion of a single gene, the gene encoding Spo0A, increased the growth-rate of M. thermoacetica. Furthermore, the gene was deleted by replacing it with a gene coding for an antibiotic resistance protein under the control of a house-hold promoter. Such alterations typically slow down growth of the modified organisms but in this case the opposite effect was seen. Moreover, contrary to what has been indicated in previous reports (see Background), in M. thermoacetica, deletion of spo0A did not result in diminished sporulation. This finding suggests that the increased growth-rate which was observed upon deletion of spo0A in M. thermoacetica was not due to a reduction in the metabolic burden related to the sporulation cascade, since the cascade was still functional.

As described in Example 2, a V198F mutation in SinR decreased the duration of the lag phase (it led to a more rapid recovery from resting state) of M. thermoacetica upon inoculation into fresh medium after a longer incubation period (Example 2). Moreover, it was found that a V198 mutation such as V198F in M. thermoacetica SinR may affect the stability of the protein, its affinity for the anti-repressor SinI and/or its ability to oligomerize (see Example 2, FIG. 4 and Tables 4 and 5).

Thus, the present inventors have identified methods for enhancing the growth (by increasing the growth-rate and/or decreasing the duration of the lag phase) of M. thermoacetica bacteria. In M. thermoacetica, there is a direct link between growth and fixation of CO₂. The present invention, which provides strains with enhanced growth, thereby provides strains with an enhanced fixation of CO₂. Furthermore, these strains may be modified to contain one or more enzymes for production of a biochemical of interest, thereby leading to increased production of such a biochemical.

In addition to increased CO₂ fixation and biochemical production, the advantages of using the methods according to the present invention for these purposes include the following:
The high cultivation temperature of M. thermoacetica has some advantages, as was also described in the background section, including: reduced risk of contamination, higher conversion rates, no requirement for cooling water, and significantly lower capital and operational expenditures when compared to other bio-based production processes.
Cells which are able to recover faster after being in a stressful situation are highly advantageous for use in bioreactors, as this allows a larger degree of fluctuation and gradients (nutrients, pH, and substrate) in the bioreactor.
In some embodiments, no genes or operons will need to be overexpressed, which would represent an increased metabolic burden. These engineered strains will maintain a high metabolic activity throughout the fermentation.

### Methods

In some aspects, the invention relates to methods for enhancing the growth of Moorella species bacteria by introducing genetic modifications into the bacteria to affect the expression and/or activity of Spo0A or to express a mutated variant of SinR.

The growth of the Moorella species bacteria may be enhanced either by increasing the growth-rate of the bacteria (number of cell divisions per time unit) or by decreasing the duration of a lag phase (the time it takes before bacteria start replicating, after they have adapted themselves to new external conditions), or by a combination of both. Both ways of enhancing growth also increase the fixation of CO₂, and, optionally, the production of a biochemical of interest.

### Bacterial Growth Measurements

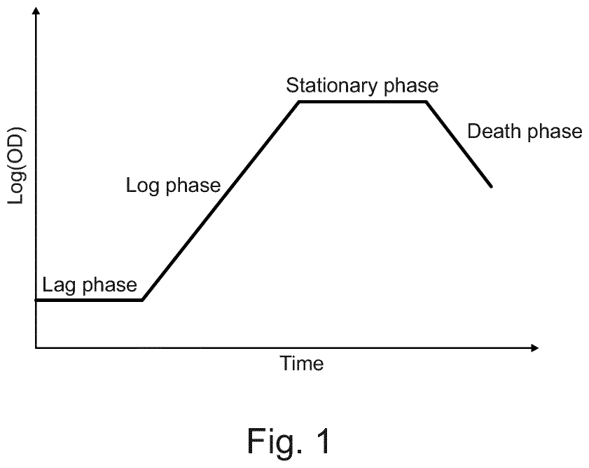

Bacterial growth is easily measured by standard techniques, including measurement of optical density (OD) at 600 nm, as used in the Examples. Continuous measurements can be used to make a graph, from which the duration of the lag phase as well as the growth-rate can be determined. To determine the duration of the lag phase, the OD of the bacterial culture (as a measure of the number of cells) should be followed from the time the cells are exposed to new external conditions, for example by being inoculated into a new medium, until the cells enter the exponential growth phase (log phase). For calculation of growth-rate, the graph is shown on a logarithmic scale (see FIG. 1). The growth-rate (p) can be calculated from two data points derived from the linear part of the graph (the exponential phase or log phase): the OD-value at time-point 1 (t₁,OD₁) and the OD-value at time-point 2 (t₂,OD₂), wherein t₂>t₁. The growth-rate can then be calculated according to formula I:

$$µ = \frac{( {log}_{10} O D 2 - {log}_{10} OD 1 ) 2.303}{t 2 - t 1} \qquad ( I )$$

### Genetic Modifications

### Spo0A

In one aspect, the invention relates to a method for increasing the growth-rate of a bacterium belonging to a Moorella species, comprising introducing one or more genetic modifications into the bacterium to reduce or abolish the expression and/or activity of Stage 0 sporulation protein A homolog (Spo0A) in the bacterium.

The expression and activity of Spo0A in such a bacterium can be determined by a person skilled in the art using standard techniques. For determination of expression levels of Spo0A mRNA or protein, techniques such as quantitative polymerase chain reaction (qPCR) and Western blot can be used. For quantifying the activity of Spo0A in the bacterium, it is first necessary to decide on which activity should be quantified. As a regulator of sporulation, Spo0A binds to DNA and controls the expression of many genes (Molle et al., Mol. Microbiol.; 50:1683-1701 2003). Therefore, the activity of Spo0A could be tested by assessing the expression of a selection of genes, including genes such as abrB, spoIIA, spoIIG, and spoIIE, e.g., by a gene microarray or using a reporter gene system containing known Spo0A binding motifs.

In some embodiments, the expression of Spo0A in such a bacterium is reduced in comparison to a control, such as, e.g., the expression level of spo0A in the bacterium prior to the introduction of the genetic modifications, the expression level in a reference bacterial cell, or a control value from, e.g., a textbook or literature. In further embodiments, the expression of Spo0A is reduced by at least 25%, such as at least 50%, such as at least 75%, such as at least 90%, such as at least 95%, in the bacterium. The expression of Spo0A may for example be reduced by knock-down of the spo0A gene, e.g., by introducing a mutation in its promoter or in the translation initiation region, such as in the ribosome binding site, by using CRISPR interference (CRISPRi), which is a CRISPR-technique using a catalytically inactive Cas enzyme, by contacting the bacterial cell with antisense sequences that interfere with transcription or translation of the gene, or by deleting a gene encoding a transcription factor which activates the transcription of spo0A or introducing a nucleic acid sequence that encodes a repressor that inhibits the transcription of spo0A.

In some embodiments, the expression of Spo0A is abolished. By that is meant that Spo0A mRNA, Spo0A protein, or both are essentially absent from, absent from or undetectable in the bacterium. The expression of Spo0A may for example be abolished by knock-out of the spo0A gene, e.g., by mutating the gene, for example by introducing a pre-mature stop-codon into the coding sequence, or by deleting the gene (which, as used herein, can mean either a partial or complete removal of the coding sequence of the gene). In some embodiments, spo0A may be knocked-out by use of technologies such as lambda Red mediated recombination, P1 phage transduction, single-stranded oligonucleotide recombineering/MAGE technologies (see, e.g., Datsenko and Wanner, 2000; Thomason et al., 2007; Wang et al., 2009) and CRISPR-based technologies. In some embodiments, spo0A may be knocked-out by transforming the bacterium with a knock-out vector and using homologous recombination to replace the gene in the chromosome, as described in Example 1. In some embodiments, the expression of Spo0A may be abolished by mutating or deleting the promoter of the gene. In some embodiments, the expression of Spo0A may be abolished using a catalytically inactive variant of CRISPR, or for example by expressing an antisense RNA that inhibits the expression or translation of Spo0A. Examples of Spo0A proteins and genes encoding them in particular Moorella species are provided herein. The endogenous gene encoding the Spo0A protein in other Moorella species, including each Moorella species specifically disclosed herein, can be identified using methods known in the art, e.g., based on gene homology.

The introduction of a vector into a bacterial host cell may, for instance, be effected by protoplast transformation (see, e.g., Chang and Cohen, Mol. Gen. Genet.; 168:111-115 1979), using competent cells (see, e.g., Young and Spizizen, J. Bacteriol.; 81:823-829 1961 or Dubnau and Davidoff-Abelson, J. Mol. Biol.; 56:209-221 1971, electroporation (see, e.g., Shigekawa and Dower, Biotechniques; 6:742-751 1988), or conjugation (see, e.g., Koehler and Thome, J. Bacteriol.; 169:5771-5278 1987).

In some embodiments, the activity of Spo0A is reduced. In further embodiments, the activity of Spo0A is reduced by at least 25%, such as at least 50%, such as at least 75%, such as at least 90%, such as at least 95%, in the bacterium. In some embodiments, the activity of Spo0A is abolished. By that is meant that one or more specific activities of Spo0A are essentially absent, absent or undetectable in the bacterium. The activity of Spo0A may for example be reduced or abolished by introducing a mutation or deletion in the coding sequence of spo0A which results in the expression of non-functional or less functional protein.

### SinR

In one aspect, the invention relates to a method for decreasing the duration of a lag phase and/or for increasing the growth-rate of a bacterium belonging to a Moorella species, comprising introducing one or more genetic modifications into the bacterium to express a variant of HTH-type transcriptional regulator SinR (SinR) in the bacterium, wherein the SinR variant has at least 90% sequence identity with SEQ ID NO: 2 and comprises an amino acid other than valine (V) at the position corresponding to position 198 in SEQ ID NO: 2, wherein the SinR variant provides for a decreased duration of the lag phase and/or an increased growth-rate of the bacterium as compared to SEQ ID NO: 2.

In some embodiments, the amino acid at the position corresponding to position 198 in SEQ ID NO: 2 is I. In some embodiments, the amino acid at the position corresponding to position 198 in SEQ ID NO: 2 is M. In some embodiments, the amino acid at the position corresponding to position 198 in SEQ ID NO: 2 is V. In some embodiments, the amino acid at the position corresponding to position 198 in SEQ ID NO: 2 is Y. In some embodiments, the amino acid at the position corresponding to position 198 in SEQ ID NO: 2 is C. In some embodiments, the amino acid at the position corresponding to position 198 in SEQ ID NO: 2 is W. In some embodiments, the amino acid at the position corresponding to position 198 in SEQ ID NO: 2 is T. In some embodiments, the amino acid at the position corresponding to position 198 in SEQ ID NO: 2 is A. In some embodiments, the amino acid at the position corresponding to position 198 in SEQ ID NO: 2 is P. In some embodiments, the amino acid at the position corresponding to position 198 in SEQ ID NO: 2 is R. In some embodiments, the amino acid at the position corresponding to position 198 in SEQ ID NO: 2 is E. In some embodiments, the amino acid at the position corresponding to position 198 in SEQ ID NO: 2 is H. In some embodiments, the amino acid at the position corresponding to position 198 in SEQ ID NO: 2 is K. In some embodiments, the amino acid at the position corresponding to position 198 in SEQ ID NO: 2 is N. In some embodiments, the amino acid at the position corresponding to position 198 in SEQ ID NO: 2 is Q. In some embodiments, the amino acid at the position corresponding to position 198 in SEQ ID NO: 2 is D. In some embodiments, the amino acid at the position corresponding to position 198 in SEQ ID NO: 2 is G. In some embodiments, the amino acid at the position corresponding to position 198 in SEQ ID NO: 2 is S.

In preferred embodiments, the amino acid at the position corresponding to position 198 in SEQ ID NO: 2 is F.

In some embodiments, the SinR variant has at least 91% sequence identity with SEQ ID NO: 2, such as 92%, such as 93%, such as 94%, such as 95%, such as 96%, such as 97%, such as 98%, and such as 99% sequence identity.

In some embodiments, a vector encoding the SinR variant is introduced into the bacterial cell by transformation, optionally using a technology described elsewhere herein. Once introduced, the gene encoding the SinR variant may be maintained as a chromosomal integrant or on a self-replicating extra-chromosomal vector.

Optionally, the endogenous sinR gene may be knocked-out, e.g., according to methods known in the art or by a method described elsewhere herein.

Optionally, the endogenous sinR gene may be left unmodified. Preferably, the endogenous sinR gene has a valine at the amino acid position corresponding to position 198 in SEQ ID NO: 2.

Preferably, for transformation of the bacterial host cell, a suitable promoter is chosen to control the expression of the SinR variant. The promoter may be native or heterologous to the bacterial host cell, i.e. it may be derived from the same species as the host cell, or it may be derived from a different species than the host cell, respectively. The promoter may be a constitutive or an inducible promoter. Constitutive promoters enable continuous protein expression whereas inducible promoters enable conditional protein expression. Using inducible promoters, protein expression may be made conditional on the presence of a specific molecule, on the presence or absence of light or on a specific temperature. Promoters that can be used to control protein expression in Moorella species bacteria include the constitutive promoter PG3PD, which is derived from M. thermoacetica and normally controls the expression of glyceraldehyde-3-phosphate dehydrogenase. Other suitable promoters are known or can be identified by the skilled person using well-known techniques.

In some embodiments, the SinR variant, comprising an amino acid other than valine (V) at the position corresponding to position 198 in SEQ ID NO: 2, may be generated by introducing one or more mutations into the gene encoding SinR on the bacterial chromosome by site-directed mutagenesis. This may for example be achieved by using homologous recombination-based techniques.

The transformation can be confirmed using methods well known in the art. Such methods include, for example, whole-genome sequencing, Northern blots or PCR amplification of DNA or mRNA, immunoblotting for expression of gene products, or other suitable analytical methods to test the presence or expression of an introduced nucleic acid sequence. Expression levels can further be optimized to obtain sufficient expression using methods well known in the art.

### Spo0A+SinR

In some aspects of the present invention, the genetic modifications relating to Spo0A and the genetic modifications relating to SinR, which modifications have been described above, are combined within the same cell. Thus, Moorella species bacteria according to the invention may, e.g., comprise both a SinR variant as described herein and lack the spo0A gene due to a deletion. Any and all aspects and embodiments relating to the various genetic modifications as described herein may be combined in any and all possible combinations.

In one aspect, the invention relates to a bacterium belonging to the M. thermoacetica and/or M. thermoautotrophica species, wherein the bacterium
(a) comprises a variant of SinR having at least 90% sequence identity with SEQ ID NO: 2 and comprising an amino acid other than V at the position corresponding to position 198 in SEQ ID NO: 2, wherein the SinR variant provides for a decreased duration of a lag phase and/or an increased growth-rate of the bacterium as compared to SEQ ID NO: 2, and
(b) has a reduced or abolished expression and/or activity of Spo0A, wherein the reduced expression and/or activity is relative to its expression and/or activity in wildtype M. thermoacetica and/or M. thermoautotrophica.

In a preferred embodiment, the spo0A gene is deleted, and the amino acid at the position corresponding to position 198 in SEQ ID NO: 2 is F.

### Genetically Modified Bacteria

In some aspects, the present invention relates to Moorella species bacteria which have been genetically modified to affect the expression and/or activity of Spo0A and/or to express a mutated variant of SinR in the bacteria.

The genetic modifications in the bacteria may be generated by techniques well known in the art and as described elsewhere herein.

In one aspect, the genetically modified bacterium may be any bacterium belonging to the genus Moorella. The species may be selected from, but is not limited to, any one of the species Moorella thermoacetica, Moorella glycerini, Moorella humiferrea, Moorella mulderi, Moorella perchloratireducens, Moorella stamsii, Moorella thermoautotrophica, Moorella sp. 215559/E30-SF1&2, Moorella sp. 60_41, Moorella sp. AIP 246.00, Moorella sp. AIP 247.00, Moorella sp. AIP 248.00, Moorella sp. AIP 383.98, Moorella sp. AIP 384.98, Moorella sp. AIP 515.00, Moorella sp. auto11, Moorella sp. auto39, Moorella sp. auto54, Moorella sp. auto59, Moorella sp. CF4, Moorella sp. CF5, Moorella sp. E306M, Moorella sp. E308F, Moorella sp. F21, Moorella sp. Hama-1, Moorella sp. HUC22-1, Moorella sp. UBA4076, Moorella sp. enrichment clone R19, Moorella sp. enrichment clone R2, Moorella sp. enrichment clone R65, Moorella sp. enrichment culture clone B1-B-65, Moorella sp. enrichment culture clone B11-B-11, Moorella sp. enrichment culture clone B13-B-103, Moorella sp. enrichment culture clone B13-B-72, Moorella sp. enrichment culture clone DGGE-band1, Moorella sp. enrichment culture clone TERIBC1, Moorella sp. enrichment culture clone TERIBC2, Moorella sp. enrichment culture clone TERIBC3, Moorella sp. enrichment culture clone TERIBC4, Moorella sp. enrichment culture clone TERIBC5, and uncultured Moorella sp. (see, e.g., the National Center for Biotechnology Information (World-Wide Web (www) address ncbi.nlm.nih.gov/Taxonomy/Browser/wwwtax.cgi?id=44260; accessed on 1 Jul. 2021).

In other aspects, the genetically modified bacterium may be any bacterium classified as belonging to the species Moorella thermoacetica and/or Moorella thermoautotrophica. A strain of M. thermoacetica may be selected from, but is not limited to, M. thermoacetica ATCC 39073 and M. thermoacetica Y72 and strains derived from any thereof, such as, for example, the M. thermoacetica strain 39073-HH. The classification of M. thermoacetica and/or M. thermoautotrophica may be based on resources such as NCBI's taxonomy browser (see the reference above), and/or it may be based on a genetic analysis.

Methods for evaluating whether two bacterial strains belong to the same or different species are known in the art and include average nucleotide identity (ANI) analysis. A specific type of ANI analysis is the ANI analysis based on MUMmer alignment (ANIm). In brief, the genome of a target strain is aligned to the genome of the reference strain, and matching regions are identified. The percentage of nucleotide identity of the matching regions are calculated as an average of all matching regions. When the comparison of two bacterial strains results in an ANIm score of at least 96.5%, they can be classified as belonging to the same species (Richter et al., PNAS; 106:19126-19131 2009, hereby incorporated by reference in its entirety).

### Use of the Genetically Modified Bacteria

In one aspect, the present invention relates to the use of the genetically modified bacteria according to aspects of the invention for metabolizing a carbon-containing substrate, optionally in the production of a biochemical.

In preferred embodiments, the carbon-containing substrate is CO and/or CO₂. Accordingly, the genetically modified bacteria as described herein can advantageously be used in methods which fixate greenhouse gases such as CO₂ which may benefit the environment.

### Carbon-Containing Substrates

Moorella species bacteria are naturally able to grow with H₂/CO₂ or CO as sole carbon source. Therefore, no further genetic modification is required to use the genetically modified bacteria according to the invention for metabolizing CO and CO₂.

Moorella species bacteria also naturally grow on other carbon-containing substrates, including xylose, fructose, methanol, glucose, arabinose, mannose, rhamnose, and pyruvate.

### Biochemicals

In embodiments of the present invention, the genetically modified bacteria according to the invention are used in the production of a biochemical.

The biochemical may, for example, be selected from C1-C4 alcohols, C1-C4 ketones, C1-C4 aldehydes, C1-C4 carboxylic acids, and any mixtures thereof. In some embodiments, the biochemical is selected from acetate, acetone, butanone, and ethanol.

For the production of a selected biochemical by the genetically modified bacteria according to the invention, the bacteria may be further genetically modified by introducing into them one or more enzymes useful for the production of the selected biochemical. Usually, the production of a biochemical requires the action of more than one enzyme; it often requires the sequential actions of a number of enzymes, constituting a specific biosynthetic pathway. The enzyme may be any characterized and sequenced enzyme, from any species, that has been reported in the literature, as long as it provides the desired activity. In some embodiments, the enzyme is an overexpressed gene which is native to the bacterium. In some embodiments, the enzyme is a functionally active fragment or variant of an enzyme which is heterologous or native to the bacterium. Also, in some embodiments, the recombinant biosynthetic pathway comprises a knock-down or a knock-out of one or more genes, typically for the purpose of avoiding competing reactions reducing the yield of the desired biochemical. To be functional in a thermophilic host cell, the enzyme should be fairly thermostable. However, it does not necessarily have to be derived from a thermophilic organism.

The introduction of the enzymes into the bacteria may occur by transforming the bacteria with one or more vectors, each encoding one or more enzymes under the control of a promoter, which, as was described for the expression of the SinR variant above, ensures expression of the genes at a suitable level so that the introduction of the genes do not overdraw substrates or energy in the host cell. The transformation may be performed as described elsewhere herein. The transformation event, introducing the enzymes for production of the selected biochemical into the cell, may optionally be combined with the introduction of any other vectors relevant to the invention, such as a knock-out vector for Spo0A and/or a vector encoding a SinR variant, as applicable. Some of the genes may be combined on the same vector.

Below are given four (preferred) examples of biochemicals which may be produced according to the invention as well as enzymes suitable for their production. The biochemicals or biosynthetic pathways should not be considered as limiting but merely as examples.

### Acetate

Being acetogens, Moorella species bacteria, including M. thermoacetica, naturally produce acetate.

### Acetone

Production of acetone in Moorella species bacteria, and more specifically in M. thermoacetica, may be enabled by the introduction of the following enzymes into the bacteria: Thiolase (ThI), Acetate acetoacetyl-CoA transferase (CtfAB), and Acetoacetate decarboxylase (Adc). See Genbank acc. number MW436696 for an example of a synthetic operon useful for acetone production in M. thermoacetica (Zeldes et al., Biotechnol. Bioeng.; 115:2951-2961 2018, Kato et al., AMB Expr.; 11:59 2021). The specific operon comprises genes encoding ThI from Caldanaerobacter subterraneus, CtfAB from Thermosipho melanesiensis, and Adc from Clostridium acetobutylicum.

### Butanone

Production of butanone in Moorella species bacteria, and more specifically in M. thermoacetica, may be enabled by introducing enzymes catalyzing the production of 2,3-butanediol and enzymes converting the resulting 2,3-butanediol to butanone into the bacteria. The 2,3-butanediol may be produced by converting pyruvate to acetolactate, which is then converted to 2,3-butanediol via acetoin, a reaction which is catalyzed by the enzymes acetolactate synthase (Als), acetolactate decarboxylase (Aldc), and 2,3-butanediol dehydrogenase (Bdh). The conversion of 2,3-butanediol to butanone may then occur by the action of a diolhydratase (pduC, pduD, and pduE) natively found in strains like Lactobacillus reuteri (Ghiaci et al., Plos One; 9(7):e102774 2014). A second way of producing butanone would be to fuse propionyl-CoA with acetyl-CoA to form 3-ketovaleryl-CoA by promiscuous β-ketothiolases and then convert the 3-ketovaleryl-CoA to butanone by the actions of an acetoacetyl-CoA:acetate/butyrate:CoA transferase (CftAB) and acetoacetate decarboxylase (Adc), commonly expressed in ABE-producing Clostridia (Srirangan et al., Biotechnology; 82:2574-2584 2016).

### Ethanol

Production of ethanol in Moorella species bacteria, and more specifically in M. thermoacetica, may be enabled either by using a bi-functional aldehyde/alcohol dehydrogenase (AdhE) enzyme converting acetyl-CoA into ethanol or by acetate reduction to acetaldehyde and further to ethanol via an aldehyde:ferredoxin oxidoreductase (AOR) enzyme and alcohol dehydrogenase (Liew et al., Metab. Eng.; 40:104-114 2017).

The invention is illustrated by the following Examples, which are not to be construed as limiting.

### Example 1

### Deletion of Spo0A Increases the Growth-Rate of M. thermoacetica

A circular knockout plasmid was constructed to delete the gene spo0A in M. thermoacetica by homologous recombination.

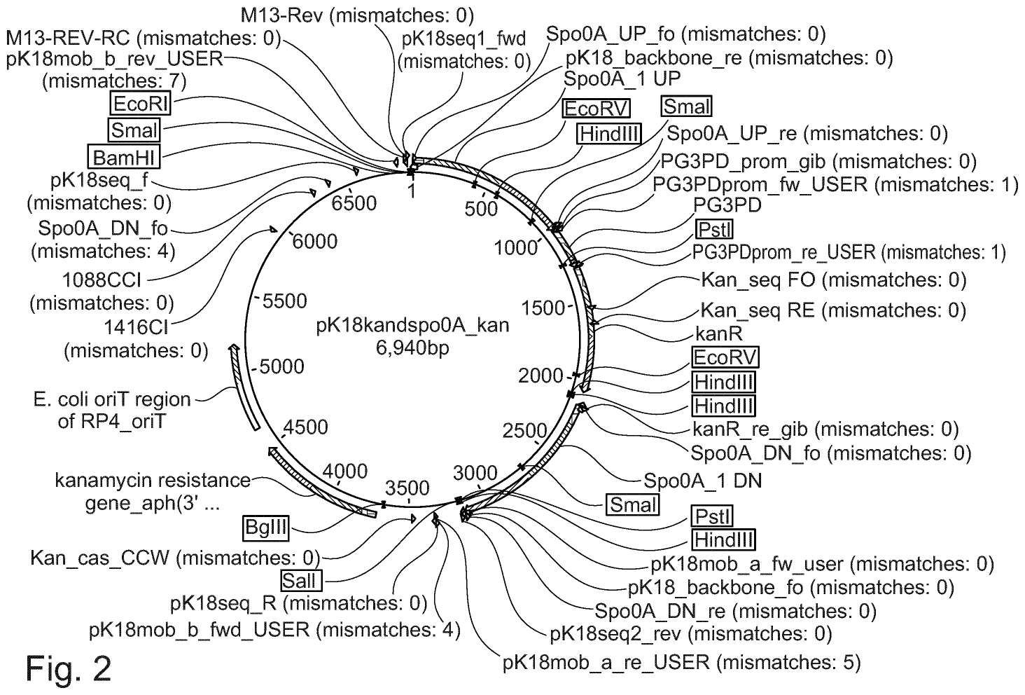

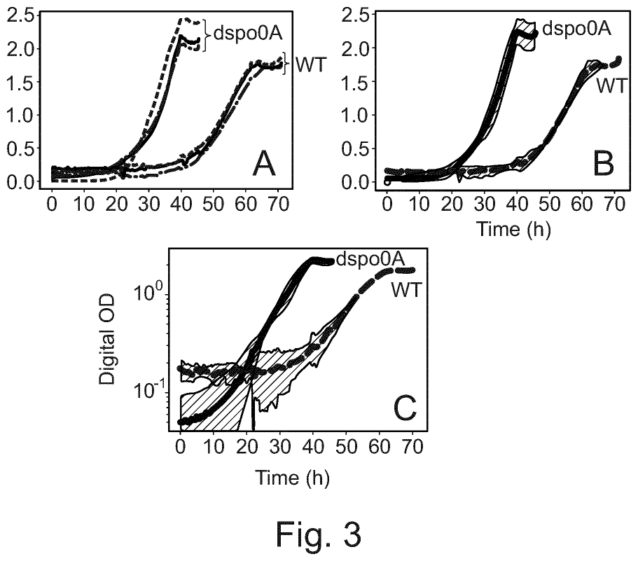

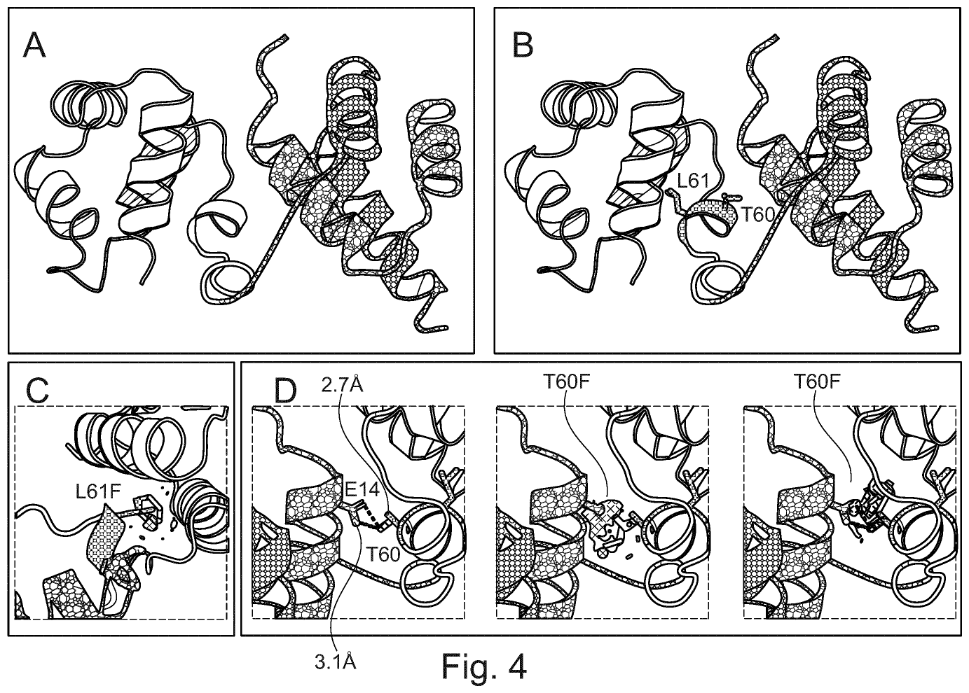

The plasmid backbone was pK18 comprising an E. coli pMB1 replicon which is not functional in M. thermoacetica and a mesophilic kanamycin resistance marker. The two homologous regions of each 1 kb up and downstream of spo0A of M. thermoacetica flanked a thermophilic kanR resistance marker under the control of the native constitutive M. thermoacetica PG3PD promoter (plasmid map is shown in FIG. 2). The plasmid was constructed by amplifying the fragments using PCR (with a high-fidelity polymerase) with the primers listed in Table 2. The fragments were assembled using the Gibson method (New England Biolabs). Once the plasmid was constructed and verified by sequencing, the plasmid was transferred to a propagation strain also assuring suitable DNA methylation.

TABLE 2
List of primers used in this experiment

| SEQ ID NO | Name | Primer sequence |
|---|---|---|
| 5 | Spo0A_UP_fo | ACGCTATAGGGGTCTTCTTG |
| 6 | Spo0A_up_re | AAAAGTTTTGGTTATCCTACACAAAATACC |
| 7 | PG3PD_prom_fo_gib | TATTTTGTGTAGGATAACCAAAACTTTTGGACGGTAAGG |
| | | ACGGTTGCCAAGTACCGGG |
| 8 | PG3PD_prom_re | TGATATTCTCATTTTAGCCATTATGTACTCCTCCTTATAT |
| | | TTATTGTAAC |
| 9 | KanR_fo | TTACAATAAATATAAGGAGGAGTACATAATGGCTAAAAT |
| | | GAGAATATCAC |
| 10 | KanR_re_gib | TTTCCCCCTCTAACCTCCTACAGTTGCGGATGTACTTCA |
| | | GAAAAG |
| 11 | Spo0A_DN_fo | AGGAGGTTAGAGGGGGAAAC |
| 12 | Spo0A_DN_re | TTAAAACCAGGGCCTTCTCC |
| 13 | pK18_backbone_fo | GGAGAAGGCCCTGGTTTTAACAGTCGACCTGCAGGCAT |
| | | GCAAGCTTGG |
| 14 | pK18_backbone_re | AAGAAGACCCCTATAGCGTAGAGGATCCCCGGGTACCG |
| 15 | Spo0A_DN_ext_250 bp | AAGCCGATAGAAAAAGAAATCCCC |
| 16 | Spo0A_UP_ext_250 bp | AATGTTACTCTACGGTGGCC |
| 17 | Kan_Seq fo | AGCAATCTGCTCATGAGTGAG |
| 18 | Kan_Seq RE | TGAAAGAGCCTGATGCACTC |

M. thermoacetica ATCC 39073 was cultivated in 100 ml serum bottles (50% filled) closed with butyl rubber stoppers (bottles and stoppers: Ochs, Germany) according to previously published methods (Daniel et al., J. Bacteriol.; 172:4464-4471 1990, Redl et al., Front. Microbiol.; 10 [3070] 2020). However, the medium was modified by replacing the buffer system with 2-(N-morpholino)ethanesulfonic acid (MES) and utilizing fructose as carbon source (60 mM final concentration). The medium had the following composition (in g/I): KH₂PO₄ (0.5); NH₄Cl (0.4); NaCl (0.4); MES (20); yeast extract (0.5); 1% trace element solution was added to the medium. The trace element solution was prepared with 2 g/l nitrilotriacetic acid; the pH adjusted to 6.0 with KOH, and the following compounds added (in mg/I): MnSO₄—H₂O (1000); Fe(SO₄)₂(NH₄)₂·6H₂O (800); CoCl₂·6H₂O (200); ZnSO₄·7H₂O (200); CuCl₂·2H₂O (20); NiCl₂·6H₂O (20); Na₂MoO₄·2H₂O (20); Na₂SeO₄ (20); Na₂WO₄ (20). The pH of the culture medium was adjusted to 6.5, flushed with N₂:CO₂ (80:20) and autoclaved at 140° C. for 40 min. The following stock solutions were added after autoclavation: CaCl₂ (50 mg/I final), MgCl₂ (330 mg/I final), vitamin solution (1%), cysteine-HCl (1 mM final). The vitamin solution contained (mg/I): biotin (2); folic acid (2); pyridoxine-HCl (10); thiamine-HCl (5); riboflavin (5); nicotinic acid (5); calcium D-(+)-pantothenate (5); vitamin B12 (0.5); p-aminobenzoic acid (5); thioctic acid (5). The medium was pre-warmed before inoculation. The strains were cultivated at 60° C. and stirred. Solid medium contained 1% Gelzan™, CaCl₂ (100 mg/I), MgCl₂ (660 mg/I), and the medium was sterilized at 120° C. for 20 min.

Prior to electroporation, cells were grown to exponential phase and harvested by centrifugation and washed twice in buffers (5 mM NaH₂PO₄/270 mM sucrose). Approximately 1 μg of plasmid-DNA was transformed into the cells by electroporation. The electroporation conditions were 1.5 kV, 500Ω by using a Bio-Rad Gene Pulser™ and a cuvette with a gap of 0.2 cm (Product of Bio-Rad Laboratories, Inc.). See Kita et al. (J. Biosci. Bioeng.; 115:347-352 2013) for more details. Recovery from electroporation was done in medium as described above but with an increased yeast extract concentration (10 g/L). The recovery was done overnight, after which 100 μl culture (in various dilutions) was plated on solid medium with 400 μg/ml kanamycin. Incubation was done anaerobically at 60° C. for 4-7 days, until colonies appeared on the plates. The colonies were tested for integration by PCR using 4 primer-sets (spo0A_up_ext_250 bp-spo0A_dn_ext_250 bp, spo0A_up_ext_250 bp-Kan_Seq re, Kan_Seq fo-spooA_dn_ext 250 bp, and Kan_Seq re-Spo0A_UP_fo (ext-ext, ext-int, int-ext, and int-int, respectively). Positive colonies were cultivated in liquid medium with 100 μg/ml kanamycin.

To further verify the transformation, gDNA was extracted from the culture and the whole genome was sequenced. This way it was confirmed that the spo0A gene was replaced by the kanR cassette.

Cultivation of the WT and Δspo0A strains was done in medium as described earlier. After entering stationary growth phase, samples were taken and inspected visually by microscope (Leica DM5000). It was clear that both cultures surprisingly formed spores. This was further confirmed by Malachite green spore staining. Malachite green 0.5% (wt/vol) in aqueous solution was added to a microscope glass slide with fixed bacteria cells. The slide was placed over boiling water to force the malachite green into the spores. After cooling (to room temperature), excess colorant was washed off by water. The stained spores were identified by microscope (Leica DM5000). In both cultures clearly green-stained spores were observed.

To further investigate the effect of the deletion, both strains were cultivated (in triplicates) with online monitoring of the optical density of the culture at 600 nm. The growth curves are shown in FIG. 3. The phenotype of M. thermoacetica Δspo0A is, surprisingly, characterized by a significantly higher growth-rate (shorter doubling time), as seen in the Figure and in the growth-rates presented in Table 3.

TABLE 3
Growth-rates of the strains

| Strain | Growth-rate |
|---|---|
| Δspo0A | 0.157 ± 0.016 h⁻¹ |
| WT | 0.104 ± 0.006 h⁻¹ |

Assessing the significance by a t-test (with confidence interval of 0.05) showed that Δspo0A grows significantly faster than the wildtype.

### Example 2

### Change in Amino Acid Sequence of SinR of M. thermoacetica

An evolution study was set up with M. thermoacetica 39073-HH to develop strains less prone to sporulation. The culture was cultivated in medium (same as described in Example 1) with 2.5 g/I yeast extract and incubated at 60° C. To apply a selection pressure, the culture was reinoculated in fresh medium once it reached stationary phase (typically after 4 days) using 2% inoculum. This approach was continued until the culture had evolved over approximately 2500 generations (14 transfers).

Characterization of the culture grown in a medium and under conditions used during the evolution was done by microscopy, malachite green staining of spores (see description in Example 1), and biomass generations (by optical density measurements). The evolved culture did not have disrupted sporulation and the generation of biomass was similar to the non-evolved strain. By storing the culture for a longer period in the incubator and recultivating the strain, it was observed that the strain was surprisingly able be become metabolically active immediately after being in a resting state for more than 25 days, in contrast to the wildtype which had a considerable lag phase; typically 1-3 days after being in resting state.

To assess the genetic changes which had occurred during the evolution, the culture was plated in various dilutions (to allow growth of single colonies) on solid medium and allowed to incubate anaerobically for 7 days at 60° C. Six single colonies were picked and cultivated in liquid medium. After 2 days of incubation, cells were spun down and genomic DNA was extracted from the individual cultures using the Wizard® Genomic DNA Purification kit (Promega, Madison, WI, United States), and extracted DNA was dissolved in 10 mM Tris-Cl, pH 8.5. Quantification of the DNA was done using the Qubit dsDNA HS Assay Kit with the Qubit 2.0 fluorometer (Thermo Fisher Scientific, Waltham, MA, United States). The DNA was used to generate Illumina shotgun sequencing libraries. Sequencing was performed by employing a MiSeq system using MiSeq Reagent Kit v3 (600 cycles), as recommended by the manufacturer (Illumina, San Diego, CA, United States), resulting in 2×300 bp paired end reads. Dominant mutations were identified by aligning to the reference genome sequence. The only mutation related to the state of the cell was a V198F mutation in the gene coding for SinR.

The evolved strain had non-disrupted sporulation, however the wildtype (carrying SinR_198V) was most prone to produce spores and the cell morphology was more round instead of distinct rods. The evolved strain with 198F had a more distinct rod shape morphology and was prone to aggregate. When used as inoculum to fresh medium, the culture with SinR_198F had a significantly shorter lag phase.

Analysis of the protein structure of SinR of M. thermoacetica shows that it resembles SinR of Bacillus, although the two proteins are not identical. Sequence alignment of the third HTH domain from SinR in M. thermoacetica and SinR from Bacillus subtilis, using two different alignment algorithms. Without being limited to theory, this suggests that V198 in M. thermoacetica may be equivalent to either T60 or L61 in B. subtilis. The SinR in B. subtilis has a defined crystalline structure which allowed for further analysis of the changes in binding to other proteins or oligomerization. Inspection of the structure of the SinR-SinI complex from B. subtilis, both the SinR oligomerization and the SinI binding (the binding of SinI mimics the oligomerization interaction (Bai et al., Genes Dev.; 7:139-148 1993, Lewis et al., J. Mol. Biol.; 283:907-912 1998)), shows that T60 and L61 are in the interphase between the HTH-domain and the oligomerization domain (FIG. 4). The sidechain of L61 is facing inside the helix-turn-helix motif, and mutation of this into a Phe (F) results in steric clashes and likely a destabilization of the protein (FIG. 4C). The sidechain of T60 is facing the oligomerization domain and makes two hydrogen bonds with E14 from SinI (FIG. 4D, left). Mutation of T60 to F would remove the hydrogen bonding capacity and result in steric clashes between SinR and SinI, most likely reducing the affinity (FIG. 4D middle and left).

The structural analysis found that L61 is facing the protein core of SinR, and mutation at L61 is therefore expected to destabilise SinR. T60 is facing the SinI interaction interphase, and mutation at this residue is therefore expected to affect the affinity of the SinR-SinI interaction. To confirm these hypotheses and identify other mutations that are predicted to have a similar effect on either SinR protein stability and/or SinI interaction affinity, two different bioinformatics tools were applied: (i) The PremPS server (Chen et al., PLOS Comp. Biol.; 16:e1008543 2020) was used to calculate the predicted effect on SinR protein stability and (ii) the mCSM-PPI2 server (Rodrigues et al., Nucleic Acids Res.; 47:W338-W344 2019) was used to calculate the predicted effect on SinI interaction affinity. In both cases, the effects of all possible mutations at both T60 and L61 were predicted.

The bioinformatic predictions for mutation at L61 (Table 4) suggest that mutation at this position has large effects on protein stability, whereas the effect on SinI interaction affinity is less pronounced. This is in agreement with the structural analysis showing that L61 is facing the protein core and does not form part of the SinI interaction site. Based on these observations, the primary contributor to any phenotypic effects observed upon mutation of L61 is expected to be protein stability. Analysis of the individual mutations at L61 show that all mutations are predicted to be destabilising with L61F being the least destabilising (predicted ΔΔG^{stability} (kcal/mol)=0.15) and L61S being the most destabilising (predicted ΔΔG^{stability} (kcal/mol)=2.77). If, as suggested by the previous sequence alignment, SinR V198 in M. thermoacetica is homologous to L61 in Bacillus, mutation of V198 to any amino acid would be expected to destabilise M. thermoacetica SinR and hence have a phenotypic effect similar to that observed for V198F.

Contrary to what was predicted for L61, mutation at T60 was predicted to have large effects on the interaction affinity between SinR and SinI, whereas the effects on protein stability were predicted to be less pronounced (Table 5). This is expected from the structural analysis, as T60 is in the SinR-SinI interaction interphase and facing the SinR surface. Considering these results, the primary phenotypic effect of mutation at T60 is expected to be due to changes in the affinity for SinI. Assessment of the individual mutations show that two mutations, T60D and T60E, are predicted to increase the affinity for SinI. This is easily explained upon inspection of the modelled mutant structures, where both the aspartate and glutamate make several new polar and hydrogen bonding contacts to SinI. The remaining part of the mutations (all other amino acids than aspartate and glutamate) are predicted to decrease the affinity for SinI. This in good agreement with the structural analysis where the sidechain of T60 was found to make two hydrogen bonds with E14 from SinI. Mutation of T60 removes the hydrogen bonding capacity, reducing the affinity between SinR and SinI. If, as suggested by the previous sequence alignment, SinR V198 in M. thermoacetica is homologous to T60 in Bacillus, mutation of V198 to any amino acid except for aspartate and glutamate would be expected to decrease the SinR-SinI interaction affinity and hence have a phenotypic effect similar to that observed for V198F.

TABLE 4
Predicted effects on protein characteristics for site-saturation
mutagenesis of L61 in Bacillus SinR

| | | | | |
|---|---|---|---|---|
| | Predicted | Predicted effect | Predicted | Predicted effect |
| | ΔΔG^{affinity} | on SinR protein | ΔΔG^{affinity} | on affinity for |
| Mutation | (kcal/mol) | stability | (kcal/mol) | SinI/R |

Stabilising mutations in SinR
None
Mildly destabilising (<1 kcal/mol) mutations in SinR

| | | | | |
|---|---|---|---|---|
| L61F | 0.15 | Neutral/Mildly | 0.69 | Increasing |

Destabilising (<1 kcal/mol) mutations in SinR

| | | | | |
|---|---|---|---|---|
| L61I | 1.17 | Destabilising | 0.5 | Increasing |
| L61M | 1.18 | Destabilising | −0.77 | Decreasing |
| L61V | 1.37 | Destabilising | −0.79 | Decreasing |
| L61Y | 1.47 | Destabilising | 0.17 | Increasing |
| L61C | 1.75 | Destabilising | −0.77 | Decreasing |
| L61W | 1.78 | Destabilising | 0.01 | Neutral |
| L61T | 2.41 | Destabilising | −0.58 | Decreasing |
| L61A | 2.53 | Destabilising | −0.85 | Decreasing |
| L61P | 2.67 | Destabilising | −0.81 | Decreasing |
| L61R | 2.68 | Destabilising | −0.59 | Decreasing |
| L61E | 2.7 | Destabilising | −0.68 | Decreasing |
| L61H | 2.7 | Destabilising | −0.50 | Decreasing |
| L61K | 2.71 | Destabilising | −0.60 | Decreasing |
| L61N | 2.72 | Destabilising | −0.52 | Decreasing |
| L61Q | 2.73 | Destabilising | −0.64 | Decreasing |
| L61D | 2.74 | Destabilising | −0.56 | Decreasing |
| L61G | 2.75 | Destabilising | −0.87 | Decreasing |
| L61S | 2.77 | Destabilising | −0.548 | Decreasing |

TABLE 5
Predicted effects on protein characteristics for site-saturation
mutagenesis of T60 in Bacillus SinR

| | Predicted | Predicted effect | Predicted | Predicted effect |
|---|---|---|---|---|
| | ΔΔG^{affinity} | on affinity | ΔΔG^{affinity} | on SinR protein |
| Mutation | (kcal/mol) | for SinI/R | (kcal/mol) | stability |
| | Mutations increasing the affinity for SinI/R | | | |
| T60D | 1.32 | Increasing | 0.12 | Neutral/Mildly |
| | | | | destabilising |
| T60E | 0.23 | Increasing | 0.07 | Neutral/Mildly |
| | | | | destabilising |
| | Mutations decreasing the affinity for SinI/R | | | |
| T60N | −1.132 | Decreasing | 0.53 | Neutral/Mildly |
| | | | | destabilising |
| T60W | −0.588 | Decreasing | 0.25 | Neutral/Mildly |
| | | | | destabilising |
| T60K | −1.182 | Decreasing | 0.58 | Neutral/Mildly |
| | | | | destabilising |
| T60P | −1.21 | Decreasing | 0.69 | Neutral/Mildly |
| | | | | destabilising |
| T60F | −0.84 | Decreasing | −0.35 | Neutral/Mildly |
| | | | | stabilising |
| T60Y | −0.58 | Decreasing | −0.93 | Neutral/Mildly |
| | | | | stabilising |
| T60C | −1.261 | Decreasing | 0.48 | Neutral/Mildly |
| | | | | destabilising |
| T60H | −0.623 | Decreasing | 0.46 | Neutral/Mildly |
| | | | | destabilising |
| T60Q | −1.184 | Decreasing | 0.33 | Neutral/Mildly |
| | | | | destabilising |
| T60G | −1.067 | Decreasing | 0.75 | Neutral/Mildly |
| | | | | destabilising |
| T60L | −1.067 | Decreasing | 0.2 | Neutral/Mildly |
| | | | | destabilising |
| T60I | −0.966 | Decreasing | 0.19 | Neutral/Mildly |
| | | | | destabilising |
| T60M | −1.136 | Decreasing | 0.16 | Neutral/Mildly |
| | | | | destabilising |
| T60K | −1.381 | Decreasing | 0.58 | Neutral/Mildly |
| | | | | destabilising |
| T60A | −0.446 | Decreasing | 0.52 | Neutral/Mildly |
| | | | | destabilising |
| T60V | −0.503 | Decreasing | 0.17 | Neutral/Mildly |
| | | | | destabilising |
| T60S | −1.143 | Decreasing | 0.28 | Neutral/Mildly |
| | | | | destabilising |

### LIST OF REFERENCES

Each reference listed below or described elsewhere herein is hereby incorporated by reference in its entirety.
Alsaker, K. V., Spitzer, T. R., and Papoutsakis, E. T. (2004). Transcriptional analysis of spo0A overexpression in Clostridium acetobutylicum and its effect on the cell's response to butanol stress. J. Bacteriol. 186(7):1959-71. doi: 10.1128/JB.186.7.1959-1971.2004.
Bai, U., Mandic-Mulec, I., and Smith, I. (1993). SinI modulates the activity of SinR, a developmental switch protein of Bacillus subtilis, by protein-protein interaction. Genes Devel. 7(1):139-148. doi: 10.1101/gad.7.1.139
Bentley, W. E., Mirjalili, N., Andersen, D. C., Davis, R. H., and Kompala, D. S. (1990). Plasmid-encoded protein: the principal factor in the “metabolic burden” associated with recombinant bacteria. Biotechnol. Bioeng. 35:668-681.
Beste, D. J. V., Espasa, M., Bonde, B., Kierzek, A. M., Stewart, G. R., McFadden, J. (2009). The genetic requirements for fast and slow growth in Mycobacteria. PLoS ONE 4(4): e5349. doi: 10.1371/journal.pone.0005349
Brown, D. P., Ganova-Raeva, L., Green, B. D., Wilkinson, S. R., Young, M., and Youngman, P. (1994). Characterization of Spo0A homologues in diverse Bacillus and Clostridium species identifies a probable DNA-binding domain. Mol. Microbiol. 14:411-426. doi: 10.1111/J.1365-2958.1994.TB02176.X
Chai, Y., Chu, F., Kolter, R., and Losick, R. (2008). Bistability and biofilm formation in Bacillus subtilis. Mol Microbiol. 67(2):254-263. doi: 10.1111/j.1365-2958.2007.06040.x
Chai, Y., Kolter, R., and Losick, R. (2009). Paralogous anti-repressors acting on the master regulator for biofilm formation in Bacillus subtilis. Mol Microbiol. 74:876-887. doi: 10.1111/j.1365-2958.2009.06900.x
Chai, Y., Norman, T., Kolter, R., and Losick, R. (2010). An epigenetic switch governing daughter cell separation in Bacillus subtilis. Genes Dev. 24:754-765. doi: 10.1101/gad.1915010
Dawson, L. F., Valiente, E., Faulds-Pain, A., Donahue, E. H., and Wren, B. W. (2012). Characterisation of Clostridium difficile biofilm formation, a role for Spo0A. PLoS One. 7:e50527.
Diallo, M., Kengen, S. W. M., and López-Contreras, A. M. (2021). Sporulation in solventogenic and acetogenic clostridia. Appl. Microbiol. Biotechnol. 105:3533-3557.
Fischer, E., and Sauer, U. (2005). Large-scale in vivo flux analysis shows rigidity and suboptimal performance of Bacillus subtilis metabolism. Nat. Genet. 37(6):636-640. issn: 10614036. doi: 10.1038/ng1555
Harris, L. M., Welker, N. E., and Papoutsakis, E. T. (2002). Northern, morphological, and fermentation analysis of spo0A inactivation and overexpression in Clostridium acetobutylicum ATCC 824. J. Bacteriol. 184(13):3586-3597. doi: 10.1128/JB.184.13.3586
Jones, S. W., Paredes, C. J., Tracy, B., Cheng, N., Sillers, R., Senger, R. S., and Papoutsakis, E. T. (2008). The transcriptional program underlying the physiology of clostridial sporulation. Genome Biol. 9(7). issn: 14747596. doi: 10.1186/gb-2008-9-7-r114
Karlin, S., Mrazek, J., Campbell, A., and Kaiser, D. (2001). Characterizations of highly expressed genes of four fast-growing bacteria. J. Bacteriol. 183:5025-5040. doi: 10.1128/jb.183.17.5025-5040.2001
Kearns, D. B., Chu, F., Branda, S. S., Kolter, R., and Losick, R. (2005). A master regulator for biofilm formation by Bacillus subtilis. Mol. Microbiol. 55:739-749. doi: 10.1111/j.1365-2958.2004.04440.x
Kiriukhin, M., and Tyurin, M. (2013). Expression of amplified synthetic ethanol pathway integrated using Tn7-tool and powered at the expense of eliminated pta, ack, spo0A and spo0J during continuous syngas or CO2/H2 blend fermentation. J. Appl. Microbiol. 114:1033-1045. doi: 10.1111/jam.12123
Lewis, R. J., Brannigan, J. A., Offen, W. A., Smith, I., and Wilkinson, A. J. (1998). An evolutionary link between sporulation and prophage induction in the structure of a repressor:anti-repressor complex. J. Mol. Biol. 283(5):907-912. doi: 10.1006/jmbi.1998.2163
Milton, M. E., Draughn, G. L., Bobay, B. G., Stowe, S. D., Olson, A. L., Feldmann, E. A., Thompson, R. J., Myers, K. H., Santoro, M. T., Kearns, D. B., and Cavanagh, J. (2020). The solution structures and interaction of SinR and SinI: Elucidating the mechanism of action of the master regulator switch for biofilm formation in Bacillus subtilis. J. Mol. Biol. 432(2):343-357. doi: 10.1016/j.jmb.2019.08.019
Molle, V., Fujita, M., Jensen, S. T., Eichenberger, P., González-Pastor, J. E., Liu, J. S., and Losick, R. (2003). The Spo0A regulon of Bacillus subtilis. Mol. Microbiol. 50(5):1683-1701. issn: 0950382X. doi: 10.1046/j.1365-2958.2003.03818.x
Pettit, L. J., Browne, H. P., Yu, L., Smits, W. K., Fagan, R. P., Barquist, L., et al. (2014). Functional genomics reveals that Clostridium difficile Spo0A coordinates sporulation, virulence and metabolism. BMC Genomics. 15:160.
Redl, S., Poehlein, A., Esser, C., Bengelsdorf, F. R., Jensen, T. Ø., Jendresen, C. B., Tindall, B. J., Daniel, R., Durre, P., and Nielsen, A. T. (2020). Genome-based comparison of all species of the genus Moorella, and status of the species Moorella thermoacetica and Moorella thermoautotrophica. Front. Microbiol. 10:3070.
Sandoval, N. R., Venkataramanan, K. P., Groth, T. S., and Papoutsakis, E. T. (2015). Whole-genome sequence of an evolved Clostridium pasteurianum strain reveals Spo0A deficiency responsible for increased butanol production and superior growth. Biotechnol. Biofuels 8:227. doi: 10.1186/s13068-015-0408-7
Seo, S., Wang, Y., Lu, T., Jin, Y., and Blaschek, H. P. (2016). Characterization of a Clostridium bejerinckii spo0A mutant and its application for butyl butyrate production. Biotechnol. Bioengineer. 114:106-112. doi: 10.1002/BIT.26057
Shi, L., Derouiche, A., Pandit, S., Rahimi, S., Kalantari, A., Futo, M., Ravikumar, V., Jers, C., Mokkapati, V. R. S. S., Vlahoviček, K., and Mijakovic, I. (2020). Evolutionary analysis of the Bacillus subtilis genome reveals new genes involved in sporulation. Mol. Biol. Evol. 37(6):1667-1678. doi: 10.1093/molbev/msaa035
Smith, M. A., and Bidochka, M. J. (1998). Bacterial fitness and plasmid loss: the importance of culture conditions and plasmid size. Can. J. Microbiol. 44:351-355.
Spigelman, G., Hoy, B. V., Perego, M., Day, J., Trach, K., and Hoch, J. A. (1990) Structural alterations in the Bacillus subtilis Spo0A regulatory protein which suppress mutations at several spo0 loci. J. Bacteriol. 172(9):5011-5019. doi: 0021-9193/90/095011-09$02.00/0
van der Veen, D., Lo, J., Brown, S. D., Tschaplinski, T. J., Martin, M., Engle, N. L., van den Berg, R. A., Argyros, A. D., Caiazza, N. C., Guss, A. M., and Lynd, L. R. (2013). Characterization of Clostridium thermocellum strains with disrupted fermentation end-product pathways. J. Ind. Microbiol. Biotechnol. 40:725-734. doi: 10.1007/s10295-013-1275-5
Zingaro, K. A., and Papoutsakis, E. T. (2012). Toward a semisynthetic stress response system to engineer microbial solvent tolerance. mBio. 3(5):e00308-12. doi: 10.1128/mBio.00308-12
WO 2009/137778 A2 (Univ. Northwestern)
WO 2010/052499A1 (TMO Renewables Ltd.)
WO 2010/098679 A1 (Lanzatech New Zealand Ltd.)
WO 2011/019717 A1 (Mascoma Corp.)
WO 2020/157487 A2 (Univ. of Nottingham)
DATABASE UniProt [Online] 24 Jan. 2006 (2006-01-24), “SubName: Full=Transcriptional regulator, XRE family {ECO:00003131 EMBL:ABC19929.1};”, XP55887769, retrieved from EBI accession no. UNIPROT:Q2R110. Database accession no. Q2R110
DATABASE EMBL [Online] 12 Jun. 2019 (2019-06-12), “Moorella thermoacetica HTH-type transcriptional regulator SinR ID—QDA00892; SV 1; linear; genomic DNA; STD; PRO; 903 BP.”, XP55887763, retrieved from EBI accession no. EMBL:QDA00892
DATABASE UniProt [Online] 1 Oct. 1996 (1996-10-01), “RecName: Full=Stage 0 sporulation protein A homolog;”, XP55887802, retrieved from EBI accession no. UNIPROT:P52941. Database accession no. P52941

1. A method for increasing the growth-rate of a bacterium belonging to a Moorella species, comprising introducing one or more genetic modifications into the bacterium to reduce or abolish the expression and/or activity of Stage 0 sporulation protein A homolog (Spo0A) in the bacterium.

2. The method according to claim 1, wherein the one or more genetic modifications comprise a genetic modification which reduces or abolishes the expression of Spo0A protein in the bacterium.

3. The method according to claim 1, wherein the spo0A gene is deleted.

4. The method according to claim 1, further comprising introducing one or more genetic modifications into the bacterium to express a variant of SinR in the bacterium, wherein the SinR variant has at least 90% sequence identity with SEQ ID NO: 2 and comprises an amino acid other than V at the position corresponding to position 198 in SEQ ID NO: 2, preferably wherein said amino acid is F, I, Y, or W, more preferably wherein said amino acid is F, and wherein the SinR variant provides for a decreased duration of the lag phase and/or an increased growth-rate of the bacterium as compared to SEQ ID NO: 2.

5. (canceled)

6. The method according to claim 4, wherein the amino acid at the position corresponding to position 198 in SEQ ID NO: 2 is phenylalanine (F), isoleucine (I), tyrosine (Y), or tryptophan (W).

7. The method according to claim 6, wherein the amino acid at the position corresponding to position 198 in SEQ ID NO: 2 is F.

8. The method according to claim 1, wherein the Moorella species is selected from
(a) Moorella thermoacetica;
(b) Moorella thermoautotrophica;
(c) a bacterial strain having an average nucleotide identity based on MUMmer alignment (ANIm) score of at least about 96.5% compared to M. thermoacetica strain DSM 512^{T};
(d) a bacterial strain having an average nucleotide identity based on MUMmer alignment (ANIm) score of at least about 96.5% compared to M. thermoacetica strain DSM 2955^{T}; and
(e) a combination of (a) and (b); (a) and (c); (a) and (d); (a), (b) and (c), or all of (a) to (d).

9. A genetically modified bacterium obtained or obtainable by the method of claim 1.

10. A bacterium belonging to the M. thermoacetica and/or M. thermoautotrophica species, wherein the bacterium has been genetically modified to reduce or abolish the expression and/or activity of Spo0A in the bacterium, wherein the reduced expression and/or activity is relative to its expression and/or activity in wildtype M. thermoacetica and/or M. thermoautotrophica.

11. (canceled)

12. A bacterium belonging to the M. thermoacetica and/or M. thermoautotrophica species, wherein the bacterium
comprises a variant of SinR having at least 90% sequence identity with SEQ ID NO: 2 and comprising an amino acid other than V at the position corresponding to position 198 in SEQ ID NO: 2, wherein the SinR variant provides for a decreased duration of a lag phase and/or an increased growth-rate of the bacterium as compared to SEQ ID NO: 2, and
has a reduced or abolished expression and/or activity of Spo0A, wherein the reduced expression and/or activity is relative to its expression and/or activity in wildtype M. thermoacetica and/or M. thermoautotrophica.

13. The bacterium according to claim 10, wherein the spo0A gene is deleted.

14. The bacterium according to claim 12, wherein the amino acid at the position corresponding to position 198 in SEQ ID NO: 2 is F.

15. A method of metabolizing a carbon-containing substrate, the method comprising contacting the carbon-containing substrate with bacterium according to claim 10.

16. The method use according to claim 15, wherein
the carbon-containing substrate is CO and/or CO₂.

17. The method according to claim 1, wherein the bacterium is of the M. thermoacetica ATCC 39073 strain or a strain derived therefrom.

18. The bacterium according to claim 10, wherein the bacterium is of the M. thermoacetica ATCC 39073 strain or a strain derived therefrom.

19. The use according to claim 16, wherein the bacterium is of the M. thermoacetica ATCC 39073 strain or a strain derived therefrom.

20. The method of claim 15, wherein metabolizing the carbon-containing substrate results in production of a biochemical.

21. The method of claim 20, wherein the biochemical is selected from a C1-C4 alcohol, a C1-C4 ketone, a C1-C4 aldehyde, a C1-C4 carboxylic acid, and any mixture thereof.


SEQUENCE LISTING (20 sequences)

SEQ ID NO: 1 - DNA, 903 bp, Moorella thermoacetica
  source 1..903: mol_type = genomic DNA
atgaccataggtgagaaactaaaaaagctaagggaagataggggaatacccctggaggaactggccgatcgcctggatatcgcccccgcctgtatggtggaagtagagcagggcaccaggaggctgtccctggccaccctgaaggaggtggctgccatcctgggggtagatgttagctattttcaagaggaaaatgataacaagaacgacaacagcgtaggcgccaggctacggcgtttgcgggagcaaaagggcattaccctgagtgaattgagccgccgttctggggtttccctggcccatatcagtgagatcgaacgctcgcgctcgacggcctcccttaagaccctggaaaaactggccgccgtcctggaggtttcaaccagttccctgttgcgctccggccagcaggattccctgggagccaagctgaagcgcctgcgggaaaagatcggcctgacccagaaggagctggcccagcaggtcggaatttcccacagcctcatcggccagattgagacagatcgcatccagccttccctgtccaccctgagtagcctggccgaggccctgggggtttctacttgttacgtccttatggaggatgacgaagaagatctctacctggattatcgcctggaggctgatatccgggaagccctgcggcggcctttgctccagcaggtcgtgcgcgacctggccggctggagcgaccgggagatcaaggggttactagattttgtaagtataatgaatgaggcccgccgcccgctaaatgacgacgatgaagaagagtaccaggaaataaaggatttcattagacgctctaaggaagaagagaggaacctggtagcaaaccttatcggggtactaaacaagaagcaccaggaggatggagtgtag

SEQ ID NO: 2 - protein, 300 aa, Moorella thermoacetica
MTIGEKLKKLREDRGIPLEELADRLDIAPACMVEVEQGTRRLSLATLKEVAAILGVDVSYFQEENDNKNDNSVGARLRRLREQKGITLSELSRRSGVSLAHISEIERSRSTASLKTLEKLAAVLEVSTSSLLRSGQQDSLGAKLKRLREKIGLTQKELAQQVGISHSLIGQIETDRIQPSLSTLSSLAEALGVSTCYVLMEDDEEDLYLDYRLEADIREALRRPLLQQVVRDLAGWSDREIKGLLDFVSIMNEARRPLNDDDEEEYQEIKDFIRRSKEEERNLVANLIGVLNKKHQEDGV

SEQ ID NO: 3 - DNA, 756 bp, Moorella thermoacetica
  source 1..756: mol_type = genomic DNA
ttagtttatcttagccccgatacgtagtttatcggcgaccatggcgataaattccgagttggtaggtttaccccgttcaacgttaatagtataaccgaagaatttattcatcatctcaacattgcccctgtcccaggcgagttcaatggcatgccggatagcgcgttcgacgcgactgggagtagtcatatacttgcgggcgataaggggatagagttccttcgttaccgctcccaggaggctgacatcgtcgataaccatcaagatagcctcccgcaggtactgataacctttgatatgggcagggacgcccatttgatggataatcttggttacctcgacatccatattacgaccggcccggctaacctgagacggcgccgtcggcaggggatggccgttggccagttgccgtaatctgtttcccagcacttccaggtcgaagggttttaatatgtagtaatcagcccccagttcgaccgaccgcatggtcatactctcctgacccagggcggtgaggataacaatcttgggtcgcggctcgaaatttaaaacctgcaatttttccaacaccccgataccgtccaggtggggcataataatatccagtataacaatatccggctcttgttcctggataagttccagggtttcctggccgttatgggctacccctgataggacgaaatcctcttgttcgttgaagtattcctccaatacctcgcaaaattcccgattatcatcggcaataagcac

SEQ ID NO: 4 - protein, 251 aa, Moorella thermoacetica
MLIADDNREFCEVLEEYFNEQEDFVLSGVAHNGQETLELIQEQEPDIVILDIIMPHLDGIGVLEKLQVLNFEPRPKIVILTALGQESMTMRSVELGADYYILKPFDLEVLGNRLRQLANGHPLPTAPSQVSRAGRNMDVEVTKIIHQMGVPAHIKGYQYLREAILMVIDDVSLLGAVTKELYPLIARKYMTTPSRVERAIRHAIELAWDRGNVEMMNKFFGYTINVERGKPTNSEFIAMVADKLRIGAKIN

SEQ ID NO: 5 - DNA, 20 bp, synthetic construct
  misc_feature 1..20: note = Primer sequence
  source 1..20: mol_type = other DNA
acgctataggggtcttcttg

SEQ ID NO: 6 - DNA, 30 bp, synthetic construct
  misc_feature 1..30: note = Primer sequence
  source 1..30: mol_type = other DNA
aaaagttttggttatcctacacaaaatacc

SEQ ID NO: 7 - DNA, 58 bp, synthetic construct
  misc_feature 1..58: note = Primer sequence
  source 1..58: mol_type = other DNA
tattttgtgtaggataaccaaaacttttggacggtaaggacggttgccaagtaccggg

SEQ ID NO: 8 - DNA, 50 bp, synthetic construct
  misc_feature 1..50: note = Primer sequence
  source 1..50: mol_type = other DNA
tgatattctcattttagccattatgtactcctccttatatttattgtaac

SEQ ID NO: 9 - DNA, 50 bp, synthetic construct
  misc_feature 1..50: note = Primer sequence
  source 1..50: mol_type = other DNA
ttacaataaatataaggaggagtacataatggctaaaatgagaatatcac

SEQ ID NO: 10 - DNA, 45 bp, synthetic construct
  misc_feature 1..45: note = Primer sequence
  source 1..45: mol_type = other DNA
tttccccctctaacctcctacagttgcggatgtacttcagaaaag

SEQ ID NO: 11 - DNA, 20 bp, synthetic construct
  misc_feature 1..20: note = Primer sequence
  source 1..20: mol_type = other DNA
aggaggttagagggggaaac

SEQ ID NO: 12 - DNA, 20 bp, synthetic construct
  misc_feature 1..20: note = Primer sequence
  source 1..20: mol_type = other DNA
ttaaaaccagggccttctcc

SEQ ID NO: 13 - DNA, 48 bp, synthetic construct
  misc_feature 1..48: note = Primer sequence
  source 1..48: mol_type = other DNA
ggagaaggccctggttttaacagtcgacctgcaggcatgcaagcttgg

SEQ ID NO: 14 - DNA, 38 bp, synthetic construct
  misc_feature 1..38: note = Primer sequence
  source 1..38: mol_type = other DNA
aagaagacccctatagcgtagaggatccccgggtaccg

SEQ ID NO: 15 - DNA, 24 bp, synthetic construct
  misc_feature 1..24: note = Primer sequence
  source 1..24: mol_type = other DNA
aagccgatagaaaaagaaatcccc

SEQ ID NO: 16 - DNA, 20 bp, synthetic construct
  misc_feature 1..20: note = Primer sequence
  source 1..20: mol_type = other DNA
aatgttactctacggtggcc

SEQ ID NO: 17 - DNA, 21 bp, synthetic construct
  misc_feature 1..21: note = Primer sequence
  source 1..21: mol_type = other DNA
agcaatctgctcatgagtgag

SEQ ID NO: 18 - DNA, 20 bp, synthetic construct
  misc_feature 1..20: note = Primer sequence
  source 1..20: mol_type = other DNA
tgaaagagcctgatgcactc

SEQ ID NO: 19 - protein, 10 aa, synthetic construct
  REGION 1..10: note = Artificial sequence
GSTDYTQNWA

SEQ ID NO: 20 - protein, 10 aa, synthetic construct
  REGION 1..10: note = Artificial sequence
GSTGYTQAWA